(19) United States
(12) Patent Application Publication
(10) Pub. No.: US 2023/0135090 A1
(43) Pub. Date: May 4, 2023

(54) METHOD FOR METERING WATER USAGE OF TAP WATER AND APPARATUS USING THE SAME

(71) Applicant: GEOGRID Inc., Seoul (KR)

(72) Inventor: Ki Hyun Kim, Namyangju-si (KR)

(21) Appl. No.: 17/551,314

(22) Filed: Dec. 15, 2021

(30) Foreign Application Priority Data
Oct. 28, 2021 (KR) 10-2021-0145541

(51) Int. Cl. G01D 4/00; G01F 1/56

(52) U.S. Cl. CPC G01D 4/004; G01F 1/56; G01D 2204/40

(57) ABSTRACT

This present disclosure presents a method for metering the amount of tap water usage, while a tap water metering device including multiple measuring sensors is supplied with power through a constant power source, including a step of the tap water metering device receiving tap water from a distribution pipe, a step of the tap water metering device metering the amount of tap water delivered to each of multiple households through a meter in the process of delivering the received tap water to multiple households, and a step of the tap water metering device transmitting the data of multiple meter readings obtained from the multiple meters to the analysis server via telecommunication.

### CROSS-REFERENCE TO RELATED APPLICATIONS

This application claims the priority of Korean Patent Application No. 10-2021-0145541 filed on Oct. 28, 2021, in the Korean Intellectual Property Office, the disclosure of which is hereby incorporated by reference in its entirety.

### TECHNICAL FIELD

The present disclosure relates to methods for metering the usage of tap water comprising: the step of the water metering device receiving tap water from distribution pipe while water metering device including plural measuring sensors is powered by a constant power source; the step of the water metering device measuring the amount of tap water delivered to each of the multiple households through multiple meters while delivering the delivered tap water to multiple households; and the step of the water metering device transmitting multiple meter reading data obtained from multiple meters to the analysis server via telecommunication.

### BACKGROUND

The Smart Water Grid, a new integrated water management system, is needed to manage the water shortage issue and to resolve environmental issues caused by climate change, industrialization, and urbanization. However, it is difficult to apply the Smart Water Grid to the conventional water meter management method.

Particularly, there was a problem that it must be replaced periodically because energy was supplied through the battery. Additionally, it must use LPWA, which has limitations in transmission speed and data, and the number of meter readings must be limited as well to maximize battery life. This method causes difficulty in reading the source data in real-time to build big data. Thus, a method is necessary to measure the amount of tap water usage in real-time and overcome social issues by constructing big data for efficient water management.

Consequently, the present disclosure intends to propose a method that can efficiently conduct remove meter reading by combining various ICT devices (sensors) with a protective case that protects the water meter and apparatus using the same.

### SUMMARY OF THE INVENTION

### Challenge to be Solved

The present disclosure is directed to solve all of the above issues.

The present disclosure is also directed to build tap water-related big data for multiple households through real-time meter reading of each household's tap water usage.

In addition, the present disclosure is directed to solve issues expected for each household based on the stored tap water usage data.

In addition, the present disclosure is directed to manage tap water use based on stored water quality data by utilizing AI.

### Means to Solve the Challenge

The characteristic configuration of the present disclosure to solve the challenges described above and realize the characteristic effects of it is as follows.

According to an exemplary embodiment of the present disclosure, a method for measuring the amount of tap water usage, may include the step of the above tap water usage meter device receiving tap water from the distribution pipe while the tap water metering device including multiple measurement sensors is supplied electricity through a constant power source instead of a temporary battery, the step of the tap water usage meter device delivering the delivered tap water to multiple households separately and metering the amount of delivered tap water to each household through multiple meters, and the step of the tap water usage meter device transmitting multiple meter reading data obtained from the multiple meters to the analysis server via telecommunication.

In addition, an exemplary embodiment of the present disclosure provides a constant power supply for supplying power for an apparatus to meter the amount of tap water usage, multiple meters measuring the amount of tap water delivered to each of multiple households in the process of receiving tap water from the distribution pipe and delivering the distributed tap water to each of multiple households, and the tap water metering apparatus including the transmitter for transmitting multiple meter reading data obtained from the multiple meters through telecommunication.

### Effects of Invention

The present disclosure can bring the following effects.

The present disclosure can construct tap water-related big data for multiple households through reading each household's tap water usage in real-time.

In addition, the present disclosure can resolve solving issues expected for each household based on the stored tap water usage.

The present disclosure can manage tap water usage based on stored water quality data by utilizing. An exemplary embodiment is to predict red tap water.

### BRIEF DESCRIPTION OF THE DRAWING

FIG. 1 shows a schematic configuration of the analysis server according to an exemplary embodiment of the present disclosure.

FIG. 2 sequentially illustrates a process until transmitting meter reading data to the analysis server according to an exemplary embodiment of the present disclosure.

FIG. 3. shows the view of the water metering device according to an exemplary embodiment of the present disclosure.

### DETAILED DESCRIPTION OF THE EMBODIMENTS

The following detailed description of the present disclosure may be understood more readily by reference to the accompanying drawings as exemplary embodiments. These embodiments are described in sufficient detail to enable those skilled in the art to practice the present disclosure. It may be understood that the various embodiments of the present disclosure are different but may not be mutually exclusive. For example, certain shapes, structures, and characteristics described herein may be implemented in other embodiments with respect to one embodiment without departing from the spirit and scope of the disclosure. In addition, it may be understood that the location or arrangement of individual components within each described exemplary embodiment may be changed without departing from the spirit and scope of the present disclosure. Thus, the following detailed description may not be taken in a limited scope, and the scope of the present disclosure, if properly described, is limited only by the appended claims, along with all scope equivalents to those claimed. Similar symbols in the drawings refer to the same or similar functions throughout the various aspects.

The present disclosure will be described more fully hereinafter with reference to the accompanying drawings, in which exemplary embodiments of the disclosure are shown, for those skilled in the art to easily practice the present disclosure.

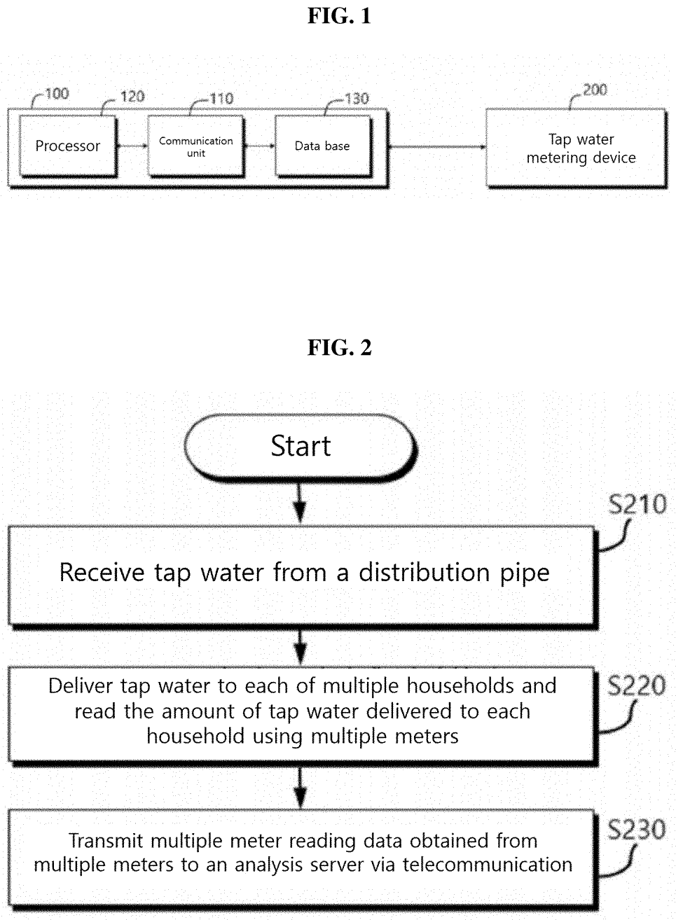

FIG. 1 shows a schematic configuration of the analysis server according to an exemplary embodiment of the present disclosure.

As illustrated in FIG. 1, the analysis server (100) of the present disclosure includes a communication unit (110) and a processor (120). In some cases, unlike FIG. 1, it may not include the database (130). For reference, the analysis server (100) may be a type of cloud server and may communicate with a tap water metering device.

The analysis server (100) may transmit and receive information to and from the tap water metering device (200) through the communication unit (110), and the communication unit (110) of the tap water metering device (200) may be implemented with various communication technologies. That is, WIFI, wideband CDMA (WCDMA), high speed downlink packet access (HSDPA), high speed uplink packet access (HSUPA), high speed packet access (HSPA), mobile WiMAX, WiBro, long term evolution (LTE), 5G, bluetooth, infrared data association (IrDA), near field communication (NFC), Zigbee, and wireless LAN technology may be applied. In addition, when a service is provided by being connected to the Internet, TCP/IP, which is a standard protocol for information transmission on the Internet, may be followed.

The database (130) of the present disclosure may store data related to tap water for each of multiple households. When using an external database, the analysis server (100) may access the external database through the communication unit (110). In addition, the analysis server (100) may communicate with the tap water metering device (200) and the terminal (300) through the communication unit (110).

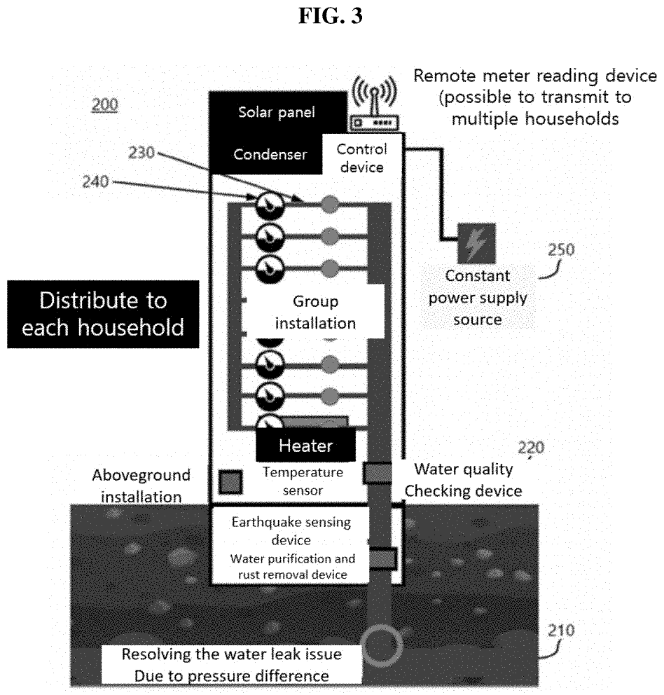

The tap water metering device (200) may include multiple measuring sensors (e.g., temperature sensor, water pressure sensor, water leak sensor, vibration sensor, earthquake sensor, EC conductivity measuring sensor, turbidity measuring sensor, and pH measuring sensor). It is connected to the distribution pipe (210) that delivers tap water and tap water flowing in from the distribution pipe (210) may be delivered to multiple households through the tap water metering device (200). The tap water metering device (200) may check tap water delivered to multiple households by using multiple measuring sensors. The tap water metering device (200) will be described later using FIG. 3.

In addition, any digital device that can communicate with a desktop computer, a notebook computer, a workstation, a PDA, a web pad, a mobile phone, a smart remote control, and various IOT main devices, has memory, and has computing power with because it is equipped with a microprocessor may become the terminal (300) of the present disclosure. The terminal (300) may correspond to a terminal of a user supplied with tap water or a terminal of a person related to the user.

That is, the processor (120) of the analysis server (100) may transmit a necessary message to a user or a person concerned through the terminal (200).

The processor (120) will be described in detail later.

FIG. 2 illustrates sequentially illustrates the process until transmitting meter reading data to the analysis server according to an exemplary embodiment of the present disclosure.

FIG. 3 shows a view of the tap water metering device according to an exemplary embodiment of the present disclosure.

The conventional tap water metering system needs to replace the battery periodically and is difficult to check it (e.g., flooding, water leakage, sanitation problems, and meter reading) real-time because the system is buried underground.

However, the tap water metering device (200) of the present disclosure overcomes the freezing issue by always maintaining the internal temperature above zero. Thus, it can be installed on the ground.

As seen in FIG. 3, the tap water metering device (200) of the present disclosure may receive necessary electricity (energy) from the constant power supply source (250) and additionally receive electricity (power) from the solar panel.

As described above, the present disclosure can measure various data in real-time or periodically by using various devices such as ICT sensors and construct related big data as it receives power from a constant power source, a solar panel, etc.

For reference, the present disclosure may read it every minute through a sensor or others and may measure approximately 1440 times per day.

The analysis server (100) may have the tap water metering device (200) acquire water quality data in real-time using multiple measuring sensors. Specifically, the tap water metering device (200) may receive tap water from the distribution pipe (210) (S210). Next, the tap water metering device (200) may deliver the received tap water to each of multiple households and may read the amount of tap water delivered to each of multiple households using multiple meters (240) (S220). The multiple measuring sensors included in the tap water metering device (200) may include an electric conductivity (EC) conductivity measuring sensor (220). The EC conductivity measuring sensor (220) may measure EC to measure ionic salts contained in tap water, and accordingly, may detect water quality in real-time.

Herein, the EC conductivity measuring sensor (220) is installed at the inside of the distribution pipe (210) or the inlet of the tap water metering device (200) connected to the distribution pipe (210). It may measure the EC of tap water based on the reference temperature periodically. Although it is indicated as a water quality checking device in FIG. 3, the EC conductivity measuring sensor (220) may be the water quality checking device.

When the EC conductivity measuring sensor (220) measures EC, a change in a temperature may affect measurements greatly. Thus, the measuring device conducts temperature correction, and it can have high accuracy in controlling the concentration of chemicals by adjusting the 0-5%/° C. slope.

Specifically, the reference temperature of the EC conductivity measuring sensor (220) is 25 degrees, and the EC of tap water can be calculated by using the following equation.

$${C}_{25} = \frac{{C}_{t}}{1 + α ( t - 25 )} \qquad Equation )$$

The C25 is the EC value at 25 degrees, while Ct stands for an EC value at t degrees and the α is a linear temperature coefficient. α may be selected between 0 and 5% per ° C., and it is usually approximately 2% per ° C. Generally, acids may have a smaller linear temperature coefficient (e.g., 1.6%), and bases may have a higher linear temperature coefficient (e.g., 2.2%).

When the EC value (based on 25 degrees) measured at the EC conductivity measuring sensor (2002) is equal to or greater than the contamination standard, the processor (120) of the analysis server (100) may prohibit the use of tap water by sending a warning message to the terminal of each household to which the tap water is delivered.

For reference, the contamination standard may vary depending on the arrival point to which tap water is delivered. Specifically, places more fatal to contamination, such as schools and hospitals, may have a higher standard value than other places, such as factories.

In addition, the tap water metering device (200) may transmit multiple meter reading data obtained from multiple meters (240) to the analysis server (100) via telecommunication (S230). The analysis server (100) may store the received meter reading data (e.g., tap water usage) in the database (130).

In addition, the analysis server (100) may receive water quality data from the tap water metering device (200) based on telecommunication and store them in the database (130). The water quality data may include the amount of tap water usage, the contamination degree of tap water, and the EC of tap water.

In addition, the tap water metering device (200) of the present disclosure may include one communication module, and the multiple meters (240) may correspond to the one communication module. The tap water metering device (200) may transmit multiple meter reading data to the analysis server (100) through telecommunication through one communication module. That is, it is a one-to-N method. For reference, the communication module included in the tap water metering device (200) may have a type of transmitter or receiver.

In addition, the transmitter of the water metering device (100) may transmit the data (e.g., meter reading data and water quality data) collected by the RS-485 (serial communication) method to the analysis server (100). Here, the RS-485 serial communication method may refer to a communication method that transmits or receives data sequentially by bit through a transmission line.

When the communication module of the tap water metering device (200) does not receive separate data from the analysis server (100), it may transmit the collected data by using the RS-485 method to the analysis server (100) according to an exemplary embodiment of the present disclosure. That is, the tap water metering device (200) and the analysis server (100) cannot transmit or receive data at the same time. When one side transmits data, the other side can only receive it.

In addition, the tap water metering device (200) may transmit data to the analysis server (100) after a predetermined time (timeout) has elapsed after receiving information from the analysis server (100). This is to prevent information loss by transmitting information from both sides at the same time.

The analysis server (100) of the present disclosure may receive information from multiple tap water metering devices (200) and manage it. Each of the multiple tap water metering devices (200) may be installed at different locations separately to read the amount of water usage for multiple households.

In addition, as described above, the multiple tap water metering devices (200) and the analysis server (100) may transmit and receive information using the RS-485 communication method. Specifically, the analysis server (100) is a kind of a main device and may transmit a command to each of the multiple tap water metering devices (200) corresponding to sub-devices.

In addition, according to an exemplary embodiment of the present disclosure, the tap water metering device (200) may collect information from the multiple meters (240) and the multiple sensors included in it by using the RS-485 communication method with each other. Moreover, it may communicate with the analysis server (100) through one communication module. Thereof, communication between the communication module of the tap water metering device (200) and the analysis server (100) may use one of various communication methods such as IoT, WCDMA, HSDPA, HSUPA, HSPA, mobile WiMAX, WiBro, LTE, 5G, bluetooth, IrDA, NFC, and Zigbee.

For reference, any one of the multiple meters (240) and multiple sensors included in the tap water metering device (200) may deliver a command to each other device corresponding to a sub-device as a main device.

Each of the multiple tap water metering devices (200) has one communication module and may communicate with the analysis server (100). The analysis server (100) and the multiple tap water metering devices (200) can communicate with a common communication line (e.g., 2-wire type and 4-wire type).

Thereof, the analysis server (main device, 100) may provide the authority to use the communication line for each of the multiple water metering devices (sub device, 200). The analysis server (100), the main device, may periodically transmit a command (e.g., to transmit collected data) to each of the sub-devices (water metering device). It will be described in more detail below. For convenience of explanation, the analysis server (100) is set as a main device and the tap water metering device (200) is set as a sub device below.

The present disclosure may assign a unique identification number to each of the sub-devices to facilitate communication. The main device may transmit a specific command to a specific sub-device (identification number is 0xfff) through the unique identification number. However, it may be required to check whether any one of the multiple sub-devices has an error.

In this case, the main device sequentially transmits a request message (e.g., reply whether the signal is good, and transmit the collected data) and receives a response message (e.g., reply message that the signal is good, and message including collected data) to or from each of the multiple sub-devices. When it does not receive a response message, it may confirm that an error has occurred in the corresponding sub-device. Thereof, the main device may wait for a response message from the sub-device for a timeout.

The timeout may vary depending on a time for receiving the collected data (e.g., meter reading data), and the timeout may be greater than the time required for receiving the collected data. This is because the main device may first receive the collected data and then receive a response message. That is, it may not determine that the sub-device is an error because it does not receive a response message while delivering the collected data.

Since the tap water metering device (200), a sub-device, reads meters of multiple households, the timeout of the tap water metering device (200) needs to be longer than the transmission time of the collected data of the household that has the largest amount of water usage among the multiple households.

Since each of the multiple tap water metering devices (200) reads a meter of a different household, the timeout for each of the multiple tap water metering devices (200) may be different from each other in some cases. Of course, multiple timeouts of the multiple tap water metering devices (200) may be longer than the transmission time of collected data of a certain household showing the largest amount of water usage among all households and may be the same.

In addition, when the main device does not receive a response message from a certain sub-device, the main device may repeat the request message for a preset number of times. In this case, the preset number of times may vary depending on the number of sub-devices, the duration of timeout, and the limit time, and it will be described with the following equation.

Number of sub-devices×duration of timeout×preset number<=limit time Equation)

For example, for convenience of explanation, the limit time, the number of sub-devices, and timeout may be set to 15 seconds, 10, and 500 msec, respectively. In this case, since 10×500 msec×the preset number is equal to or less than 15, the preset number may correspond to 3 or less. That is, the main device may repeatedly transmit the request messages 1 to 3 times.

Of course, data may be transmitted in other ways, and it may be transmitted by various technologies such as LTE and WIFI. In some cases, the tap water metering device (200) may include at least one transmitter and/or one receiver (communication module).

In addition, the processor (120) of the analysis server (100) may consistently monitor whether tap water is contaminated based on the water quality data stored in the database (120).

As shown in FIG. 3, the tap water metering device 200 of the present disclosure may include multiple meters (240). The tap water flowing from the distribution pipe (210) may be delivered to each of multiple households through the water pipe for households (230).

Each of the multiple meters (240) may check the meter reading data of tap water (e.g., the amount of tap water) flowing into each of the multiple households.

Thus, the database (130) may store the average tap water usage measured for each of multiple households (e.g., household a and household b) using multiple meters (240).

Additionally, the database (130) may store the household information for each of the multiple households (e.g., age of household members and number of household members). For example, it may store information that household a includes one male in his 70s and that household b includes one male in his 50s, one female in her 50s, one male in his 20s, and four females in their teens.

While the database (130) stores the average daily tap water usage, it can assume that the daily tap water usage of one household of a certain day (e.g., 300 L) is beyond the range of the average daily usage of tap water for a certain period (e.g., 1 week) of the household.

Thereat, the processor (120) of the analysis server (100) may transmit a warning message that the water usage is too much or too little than usual to the terminal (300) corresponding to the household. The above process may be performed using the AI module included in the processor (120) of the analysis server (100) of the present disclosure.

Thereat, the terminal (300) corresponding to the household may correspond to a terminal pre-stored in the database (130). For example, if the first household has one elderly person (a) in his 70s living alone, the terminal may correspond to the terminal of the elderly living alone (a) or the terminal of his child (a′) (or the terminal of a related department such as a group for protecting senior citizens living alone). If the tap water usage of the first household (a) is less water than usual, the child (a′) will be able to immediately check what has happened to the health of the elderly person (a) living alone and take appropriate measures.

On the other hand, if the database (130) may record that a household has four or more members and the average daily tap water usage is out of a certain range, the processor (120) of the analysis server (100) may not send a warning message unless there is a separate request. That is, whether the warning message is transmitted may be determined by the number of household members.

In addition, a certain range (based on average daily tap water usage) that is a criterion for delivering the warning message may be larger for a household with a larger number of members than a household with a smaller number of members. This is because when the number of household members is large, the range of tap water usage (small and large) may be larger.

In addition, the processor (120) of the analysis server (100) may charge the tap water usage cost of one household having the lowest average daily usage among the multiple average daily usages of multiple households at a discounted price or may support the household to be charged less. That is, it may charge the tap water usage fee directly through the analysis server (100), but it also may support other systems (e.g., water and sewerage office) to charge the tap water usage fee.

Thereat, the discount rate of the discounted usage fee may be determined based on the average (A) of the average daily tap water usage of each of the multiple households and the average daily tap water usage (B) of the household. Specifically, the discount rate may be A-B/A.

For example, if the average value (A) of the average daily tap water usage of each of the multiple households is 300 L and the average daily tap water usage (B) of the household is 200 L, the discount rate is 300-200/300=33.333% (approximately 33%).

In some cases, the processor (120) of the analysis server (100) provides a mini game to the terminal (300) of the household member of the household. The discount rate may be determined according to the result of the mini game (e.g., 33% discount, 50% discount, or no discount). This is to reduce the imprudent use of tap water and to use the server (app) of the present disclosure.

In addition, in another embodiment of the present disclosure, multiple households may be divided into two or more groups, and a household using the least amount of daily tap water usage for each group may be selected. In addition, the processor (120) of the analysis server (100) may provide a mini-game to each of the terminal (300) of the selected household and may determine to apply different discount rates depending on the results of the mini-game (e.g., 33% discount, 50% discount, or no discount).

The exemplary embodiments according to the present disclosure described above may be implemented in the form of program commands that can be executed through various computer components and recorded in a computer-readable recording medium. The computer-readable recording medium may include program commands, data files, and/or data structures, alone or in combination. The program commands recorded on the computer-readable recording medium may be specially designed and configured for the present disclosure or may be available as they are open to those skilled in the computer software field. Examples of the computer-readable recording medium include a hard disk, a magnetic medium such as a floppy disk and a magnetic tape, an optical recording medium such as a CD-ROM and DVD, and a magneto-optical medium such as a floptical disk, and hardware devices specially configured to store and execute program commands, such as ROM, RAM, flash memory. Examples of program commands include not only machine language codes such as those generated by a compiler, but also high-level language codes that can be executed by a computer using an interpreter or the like. The hardware device may be configured to operate as one or more software modules for processing according to the present disclosure, and vice versa.

In the above, the present disclosure has been described with specific matters such as definite components, limited embodiments, and drawings. They are provided to help a more general understanding of the present disclosure, and the present disclosure should not be construed as being limited to the embodiments set forth herein. Those skilled in the art may devise various modifications and variations from these descriptions.

Thus, the spirit of the present disclosure should not be limited to the above-described embodiments, and not only the claims described below but also all modifications equally or equivalently to the claims described below belong to the scope of the spirit of the present disclosure.

### DESCRIPTION OF SYMBOLS

100: Analysis server
110: Communication unit
120: Processor
130: Data base
200: Tap water metering device
210: Distribution pipe
220: Water quality checking device (e.g., EC measuring sensor)
230: Water pipe for households
240: Meter
250: Constant power supply source
300: Terminal

What is claimed is:

1. A method of metering the amount of tap water usage, comprising:
While a tap water metering device comprising multiple measuring sensors is supplied with power through a constant power source;
(a) A step of the tap water metering device receiving tap water from a distribution pipe;
(b) A step of the tap water metering device metering the amount of tap water delivered to each of multiple households through a meter in the process of delivering the received tap water to multiple households; and
(c) A step of the tap water metering device transmitting the data of multiple meter readings obtained from the multiple meters to the analysis server via telecommunication.

2. The method of metering the amount of tap water usage of claim 1,
wherein one communication module is comprised in the tap water metering device and the multiple meters correspond to the one communication module,
characterized in that the tap water metering device delivers the reading data of the multiple meters to the analysis server through telecommunication through the one communication module.

3. The method of metering the amount of tap water usage of claim 1,
wherein the multiple measuring sensor is comprised in an EC measuring sensor,
characterized in that the EC measuring sensor is installed at the inside of the distribution pipe or the inlet of the tap water metering device connected to the distribution pipe to measure the EC of the tap water based on the reference temperature periodically.

4. The EC measuring sensor of claim 3,
wherein the reference temperature is 25 degrees, and the EC of the tap water is calculated by the following equation,
$${C}_{25} = \frac{{C}_{t}}{1 + α ( t - 25 )} \qquad equation )$$
characterized in that the C25 is an EC value at 25 degrees, the Ct is an EC value at t degrees, and the α refers to a linear temperature coefficient.

5. The method of metering the amount of tap water usage of claim 1,
a method characterized in that, in the process of delivering the tap water flowing out from the distribution pipe to each of the multiple households through tap water pipe for households,
each of the multiple meters checks the water quality data of tap water flowing into each of the multiple households and the water quality data is further comprised in the amount of tap water usage, the contamination degree of tap water, and the EC of tap water,
daily average tap water usage is measured for each of the multiple households using multiple meters and stored in a database included in the analysis server, and
when the daily tap water usage of one household during a certain period exceeds the mean daily tap water usage of the household more than a certain degree, the analysis server delivers a warning message to a terminal corresponding to the household.

6. A device reading the amount of tap water usage comprising:
A constant power source to provide power;
Multiple meters reading the amount of tap water delivered to each of the multiple households in the process of receiving tap water from a distribution pipe and delivering the tap water to multiple households; and
A transmitter that transmits multiple meter reading data obtained from the multiple meters through telecommunication.
